(19) United States
(12) Patent Application Publication
(10) Pub. No.: US 2026/0060851 A1
(43) Pub. Date: Mar. 5, 2026

(54) SYSTEM AND DEVICE FOR INCONTINENCE GARMENTS

(71) Applicant: Wildhawk, Inc., San Carlos, CA (US)

(72) Inventor: Jack David Lynch, San Carlos, CA (US)

(21) Appl. No.: 19/309,954

(22) Filed: Aug. 26, 2025

Related U.S. Application Data
(60) Provisional application No. 63/687,609, filed on Aug. 27, 2024.

(51) Int. Cl. A61F 13/496; A61F 13/15; A61F 13/491

(52) U.S. Cl. CPC A61F 13/496; A61F 13/4915; A61F 2013/15121; A61F 2013/15544

(57) ABSTRACT

A garment system for incontinence management includes a pouch integrated into underwear or other close-fitting garments. The pouch comprises a front panel and a rear panel with mesh fabric, connected by vertically opposed seams and a horizontally opposed lower seam formed with a gusset. An opening between the panels allows insertion of a removable incontinence guard from the outside of the garment. The mesh fabric may be positioned against the wearer's body to efficiently wick body fluids from one side to the other, transferring moisture to the incontinence guard within the pouch. The garment may include boxer briefs, briefs, or workout clothing. This system provides improved comfort and discretion by separating the absorbent guard from direct skin contact while maintaining effective fluid management through the specialized mesh construction.

### CROSS-REFERENCE TO RELATED APPLICATIONS

This application claims priority to U.S. Provisional Application No. 63/687,609, titled System and Device for Incontinence Garments, filed Aug. 27, 2024, which is hereby incorporated by reference in its entirety.

### FIELD OF DISCLOSURE

The present disclosure relates to the field of men's incontinence products such as guards or pads to catch and hold body fluids and men's underwear.

### BACKGROUND

Existing men's incontinence guards suffer from several deficiencies. Guards generally have adhesives on one side to secure to men's underwear to hold the guard in the correct placement to absorb unwanted body fluids. Adhesives to secure guards to underwear are unreliable. The guard tends to move away from the desired placement in men's underwear because the adhesive used to fasten the guard to the underwear can unstick. Another issue is that removing an adhesive backed guard from men's underwear is noisy, and would cause unwanted attention to the user, especially in a public restroom.

Another issue is chafing. Material on the existing guards tends to bunch and gather on the sides of guards and rub the skin of the wearer in the groin and crotch area. The human skin in the groin and crotch area is sensitive and is easily chafed by the bunched and gathered guard material on the side of the guards.

Another issue is the difficulty and inconvenience with placing and replacing guards in men's underwear. Placing or replacing guards generally requires the wearer to pull down their pants and underwear to position a guard or pad properly in their underwear. This process can also be difficult and embarrassing in a public setting, such as public restroom at a urinal or toilet stall.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

In some aspects, the techniques described herein relate to a garment including: a gusset; a pouch, wherein the pouch includes: a front panel; a rear panel, wherein the rear panel includes a fabric that is different from other parts of the garment; two vertically opposed seams formed between the front panel and the rear panel; a horizontally opposed lower seam formed between the front panel, the rear panel, and the gusset; an opening formed between the front panel and the rear panel; and a removable incontinence guard, wherein the removable incontinence guard is positioned within the pouch, wherein the opening of the pouch is positioned on an outside of the garment.

In some aspects, the techniques described herein relate to a garment, wherein the fabric that is different from other parts of the garment is a mesh fabric.

In some aspects, the techniques described herein relate to a garment, wherein the mesh fabric is treated to be hydrophobic and efficiently transports body fluids from one side of the mesh fabric to the other side of the mesh fabric.

In some aspects, the techniques described herein relate to a garment, wherein one side of the mesh fabric is in at least partial communication with a human body and the mesh fabric transports body fluids from the human body.

In some aspects, the techniques described herein relate to a garment, wherein the garment includes underwear.

In some aspects, the techniques described herein relate to a garment, wherein the garment includes boxer briefs.

In some aspects, the techniques described herein relate to a garment, wherein the garment includes briefs.

In some aspects, the techniques described herein relate to a garment, wherein the garment includes workout pants or shorts.

In some aspects, the techniques described herein relate to a garment, wherein the pouch further includes a securing means positioned near the opening of the pouch.

In some aspects, the techniques described herein relate to a garment, wherein the pouch further includes an elastic band to narrow the opening of the pouch to removably retain the incontinence guard within the pouch.

In some aspects, the techniques described herein relate to a garment, wherein the front panel is substantially a same color as the removable incontinence guard.

In some aspects, the techniques described herein relate to a garment, wherein the front panel substantially prevents moisture from passing through the front panel.

In some aspects, the techniques described herein relate to a garment, wherein the fabric that is different from other parts of the garment is a provides a one-way transport of moisture away from a human body to the removable incontinence guard.

In some aspects, the techniques described herein relate to a garment, wherein the front panel includes a reinforced lip at the opening.

In some aspects, the techniques described herein relate to a garment, wherein the front panel includes two panels of fabric.

In some aspects, the techniques described herein relate to a garment, wherein the pouch is formed on the outside of the garment.

In some aspects, the techniques described herein relate to a garment, wherein the opening is located at the top of the garment.

In some aspects, the techniques described herein relate to a garment, wherein the removable incontinence guard is provided independent of a fastener to secure the removable incontinence guard inside the pouch.

In some aspects, the techniques described herein relate to a garment, wherein the pouch is tapered such that the top of the pouch is wider than the bottom of the pouch.

In some aspects, the techniques described herein relate to a garment, wherein the removable incontinence guard is tapered to substantially match with the taper of the pouch.

### BRIEF DESCRIPTION OF FIGURES

Non-limiting and non-exhaustive examples are described with reference to the following figures.

FIG. 1 shows the exterior front view of the garment of some implementations, with a representation of how the guard worn by a user.

FIG. 2 shows the exterior front view of the garment of some implementations, with a representation of how the guard is inserted into the pouch.

FIG. 3 shows the interior front view of the garment of some implementations, with a representation of how the guard is inserted into the pouch.

FIG. 4 shows a cross-section view of the garment from the left side of some implementations, with the guard inserted into the pouch.

FIG. 5 shows a view from above of the interior of the garment in some implementations, with a representation of how the guard is inserted into the pouch.

FIG. 6 illustrates a detailed view of a mesh knit structure that may be used as a portion of fabric in the garment in some implementations, showing the construction and arrangement of the mesh pattern with interconnected fibers forming openings throughout the structure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The disclosure relates to customized garments that include an exterior pouch to removably receive an incontinence guard or pad to collect body fluids. In some implementations, the incontinence guard is removably fixed within the pouch of the garments. In some implementations, the interior of the pouch facing a wearer's body and in communication with the wearer's skin can include a specialized mesh fabric panel that draws moisture away from the wear's skin to an incontinence guard or pad located within the exterior pouch. The underwear with such a pouch may include boxer briefs, regular briefs, boxers, or any other suitable type of underwear. The pouch may be installed in other types of suitable clothing that is worn next to the skin and positioned around a wearer's crotch and worn close to a wearer's skin, such as cycling pants, exercise pants, workout pants, shorts, etc. By keeping the incontinence guard separate from a wearer's skin, the customized garments with the pouch can avoid direct contact between the wearer's skin and the incontinence guard.

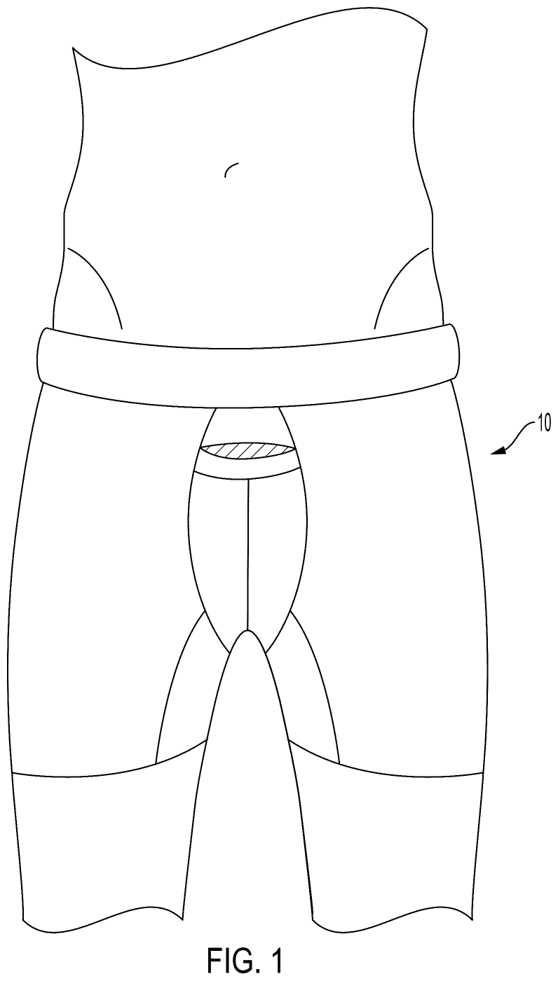

FIG. 1 illustrates some implementations of a pair of boxer briefs 10 shown from an exterior front perspective. The boxer briefs 10 may be configured with a specialized pouch system designed to accommodate an incontinence management solution. In some implementations, the boxer briefs 10 include a waistband portion at the upper section of the garment that may provide comfortable fit around a wearer's waist. The main body section of the boxer briefs 10 extends downward from the waistband and may be constructed from suitable fabric materials that provide stretch and comfort during wear as well as protection from incontinence leaks.

The boxer briefs 10 may include leg portions that extend from the main body section and may be configured to provide appropriate coverage and fit around a wearer's thighs. In some implementations, the leg portions may include hemmed edges that may help prevent rolling or bunching during use. The overall construction of the boxer briefs 10 may maintain the appearance and functionality of traditional underwear while incorporating the specialized pouch system for incontinence management.

In some implementations, the exterior view of the boxer briefs 10 in FIG. 1 may show the general silhouette and proportions of the garment when worn. The design may be configured to appear substantially similar to conventional boxer briefs from the exterior, which provides discretion for the wearer. The boxer briefs 10 may be sized and shaped to accommodate various body types and may be available in multiple sizes to ensure proper fit and comfort.

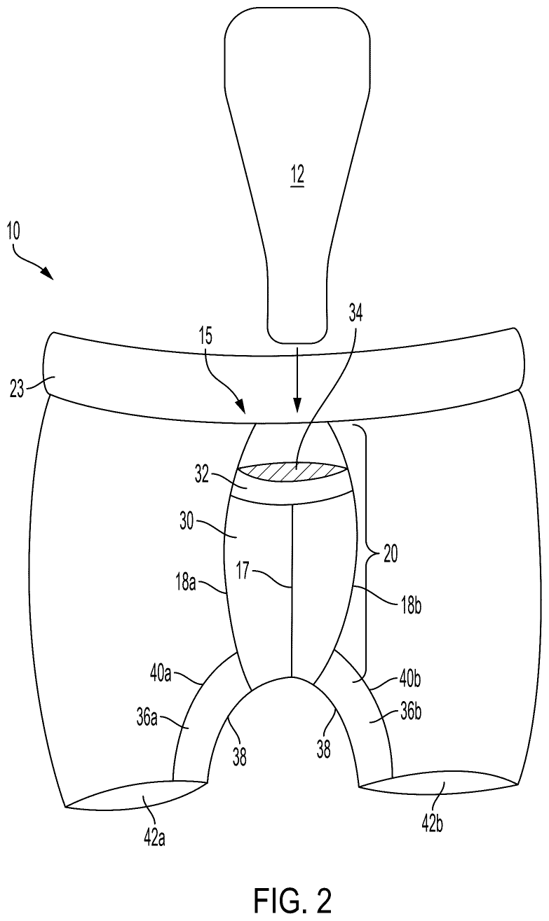

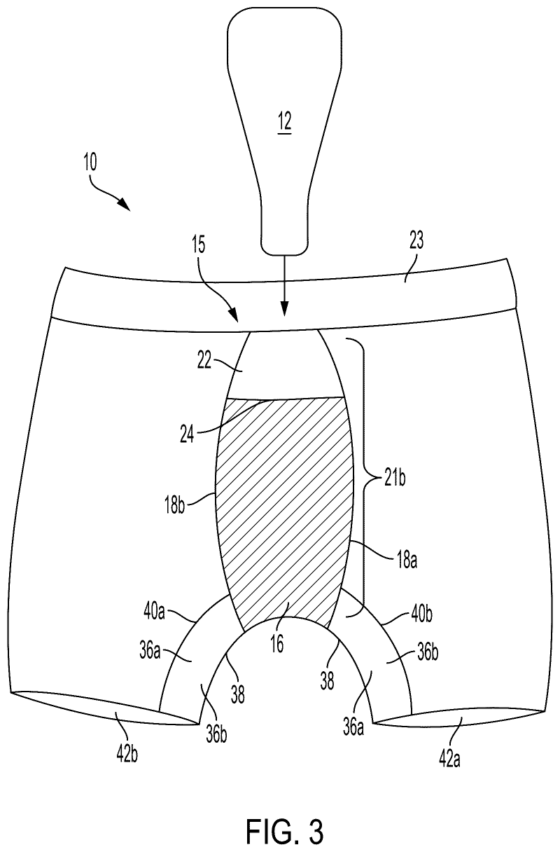

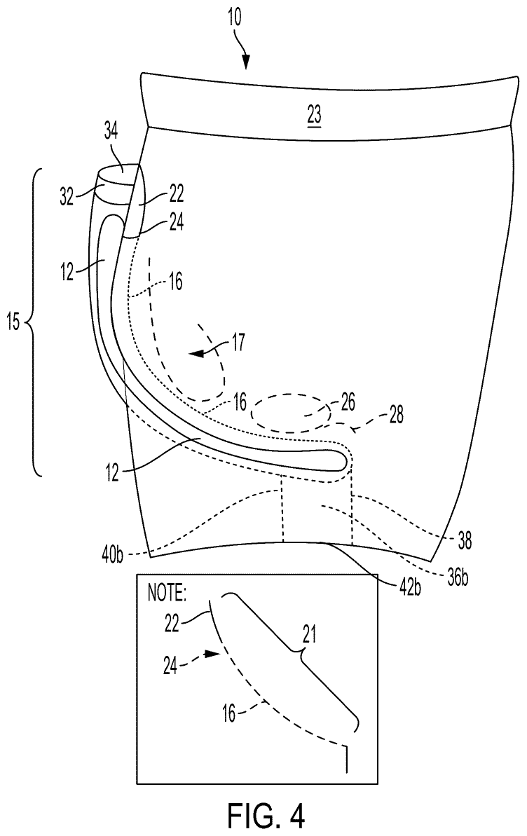

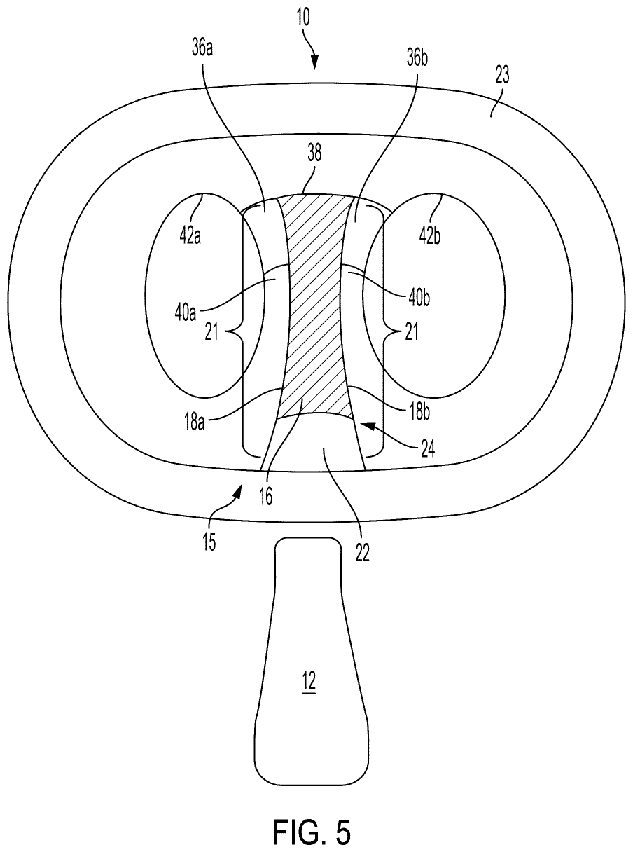

FIG. 2 illustrates some implementations of a pair of boxer briefs 10 that have been modified to include a novel pouch. The pouch 15 can be configured to securely hold a removable absorbent incontinence guard 12. In some implementations, the pouch 15 is formed by an exterior front panel of body fabric 30 and an interior panel 21 (see FIG. 3, FIG. 4, and FIG. 5). In some implementations, the exterior front panel 30 is attached to two vertical seams, right vertical seam 18a and left vertical seam 18b that frame the central section 20. In some implementations, the pouch comprises an opening at the top of the pouch to removably receive the incontinence guard 12. In some implementations, when the pair of boxer briefs 10 includes a pouch 15 with an opening at the top, the location of the opening provides a wearer with easier access to the pouch while the wearer has an additional garment worn over the pair of boxer briefs 10. For example, if the wearer has outer pants worn over the pair of boxer briefs 10, the wearer can easily access the pouch 15 to insert or remove an incontinence guard 12 without removing the outer pants or minimize the how much of the outer pants that are opened to access the pouch 15 opening. In some instances, a wearer may be enabled to access the pouch 15 opening without opening or substantially opening or substantially removing the outer pants. This top pouch opening is advantageous in a public setting, such as a public restroom, where a wearer desires to minimize unwanted attention when using an incontinence guard 12. In some implementations, the seams used for boxer briefs 10 may be constructed with flat lock stitching or flat felled seams to minimize irritation to the wearer and to provide a flexible and strong stitching. In some implementations, other suitable stitching can be implemented. In some implementations, exterior front panel 30 comprises one or more panels of fabric. In some implementations, exterior front panel 30 comprises two panels of fabric stitched together at seam 17. In some implementations, the central section 20 of boxer briefs 10 is configured to be wide enough to cover the genitalia of the wearer, with some extra margin to account for movement of the garment when the wearer is sitting or standing. In some implementations, the exterior front panel 30 extends to the rear gusset seam 38 of the right gusset section 36a and left gusset section 36b. The right gusset section 36a can be a strip of fabric on the inner right leg bordered by the following: the rear gusset seam 38 (see FIG. 3), the front right gusset seam 40a, the right vertical seam 18a framing the interior panel 21, and the hem of the right underwear leg 42a. The left gusset section 36b can be a strip of fabric on the inner left leg bordered by the following: the rear gusset seam 38, the front left gusset seam 40b, the left vertical seam 18b framing the interior panel 21, and the hem of the left underwear leg 42a.

In some implementations the exterior front panel 30 and the right gusset section 36a and left gusset section 36b can be formed from the same material as other parts of the boxer briefs 10. In some implementations, the exterior front panel 30 and the right gusset section 36a and left gusset section 36b can be formed from material that is different from the material used in other parts of the boxer briefs. For example, exterior front panel 30 may be formed from one or more materials that help block moisture transfer. In some implementations, exterior front panel 30 can be constructed from one or more layers. In some implementations, for example, exterior front panel 30 may be constructed from a waterproof material to prevent excess moisture from incontinence guard 12 from leaking to the front of a user's outer pants. In some implementations, the exterior front panel 30 may include a durable waterproof coating on the inside of the pouch facing the incontinence guard 12, which will help repel moisture from transferring from the inside of pouch 15 to the surface of exterior front panel 30 and the wearer's outer garment. In some implementations, the exterior front panel 30 may include a waterproof but breathable layer, such as a polytetrafluoroethylene based material that keeps moisture on the inside of the pouch, but still enabling the pouch to pass water vapor, such as sweat. Other suitable waterproof and breathable layers can be used with exterior front panel 30.

The top edge 32 of the exterior front panel 30, can be located a short distance below the waistband 23 and can be left unconnected to the main body of boxer briefs 10 to create an opening 34 of the pouch. In some implementations, this opening 34 is configured to removably receive the incontinence guard 12. The incontinence guard 12 and pouch 15 are designed such that the incontinence guard 12 fits securely in the pouch 15. When incontinence guard 12 is inserted into the pouch 15, the incontinence guard 12 is held securely in place by the two vertical seams right vertical seam 18a and left vertical seam 18b, the rear gusset seam 38, the exterior front panel 30, and the interior mesh 16 (see FIG. 3, FIG. 4, and FIG. 5). In some implementations, the incontinence guard 12 can be sized long enough that the pouch 15 extends from a short distance below the top edge 32 of the exterior front panel 30 to the rear gusset seam 38. In some implementations, the removable absorbent incontinence guard 12 can be disposable or reusable. In some implementations, the exterior front panel 30 can formed in a tapered shape such as a triangle or a trapezoid. In some such implementations, the top of exterior front panel 30 that meets with the top edge 32 is wider than the bottom edge of the exterior front panel 30 that meets the rear gusset seam 38. In some implementations, the tapered designs aids in holding the incontinence guard 12 from shifting within the pouch 15. In some implementations, the incontinence guard 12 is formed in a shape that is the same or similar to the shape of the pouch 15. In some implementations, the incontinence guard 12 is formed in a tapered shape such as a triangle or a trapezoid. In some implementations, the incontinence guard 12 may be scaled in size to enable the incontinence guard 12 to fit within the pouch 15. It should be appreciated that the pouch and incontinence guard 12 can be formed in other suitable shapes.

In some implementations, top edge 32 of the pouch 15 is a reinforced lip. In some implementations, the reinforced lip is a layer of the material used to form the top edge 32 that is folded over and sewn to top edge 32. In some implementations, the reinforced lip is one or more separate materials that can increase strength of the pouch opening 34, but help maintain the strength and elasticity of the pouch opening 34. In some implementations, top edge 32 may comprise additional elastic material to ensure that top edge 32 remains flexible after frequent stretching from inserting and removing incontinence guard 12 from the pouch 15. In some implementations, top edge 32 may include an elastic band to narrow the opening of the pouch 15, which can help narrow the opening of the pouch 15 to aid in retaining the incontinence guard 12. In some implementations, top edge 32 may comprise fasteners to better hold the incontinence guard 12 in the pouch 15. For example, in some implementations, top edge 32 may include snaps to securely hold the top edge 32 against apron 22. In some implementations, top edge 32 may include hook and loop closures to securely hold the top edge 32 against apron 22. In some implementations, top edge 32 may include sealed magnet (to prevent rust) closures to securely hold the top edge 32 against apron 22 (e.g., securely connecting polar opposite magnets on top edge 32 and apron 22).

FIG. 3 illustrates some implementations of a pair of boxer briefs 10 that include a novel pouch 15. FIG. 3 illustrates the pair of boxer briefs 10 turned inside out to show the interior construction of the boxer briefs 10. The pouch 15 can be configured to securely hold a removable absorbent incontinence guard 12. In some implementations, the wall of the pouch 15 that is in communication with the wearer's body is formed by an interior panel 21 and an exterior front panel of body fabric 30. The interior panel 21 is attached to two vertical seams right vertical seam 18a and left vertical seam 18b that frame the interior panel 21. While interior panel 21 and exterior front panel 30 are illustrated as sharing the right vertical seam 18a and the left vertical scam 18b, in some implementations, interior panel 21 and exterior front panel 30 are connected to the boxer briefs 10 using separate scams. For example, in some implementations, exterior front panel 30 can be made wider than interior panel 21 such that the panels do not share the same seams. In some implementations, the interior panel 21 is configured to be wide enough to cover the genitalia of the wearer, with some extra margin to account for movement of the garment when the wearer is sitting or standing. In some implementations, the interior panel 21 (or rear panel) can be divided into two subsections that are connected: an apron 22 made from one type of fabric such as the same fabric used for the main body fabric of the boxer briefs 10 and a different fabric, such as an interior mesh 16. While interior mesh 16 is described herein as a mesh, in some implementations, a different fabric can be used than a mesh fabric. As will be described in more detail below, in some implementations, the fabric for interior mesh 16 (whether a mesh fabric or other type of fabric) may comprise hydrophobic characteristics, wicking characteristics, one-way moisture transfer characteristics, or some combination of the forgoing to help keep the skin of the wearer of the boxer briefs 10 as dry as possible from incontinence leaks and sweat.

In some implementations, the apron 22 extends down from the waistband 23 for a small portion of the length of the interior panel 21. In some implementations, apron 22 can be used to increase the strength of interior panel 21 by minimizing the amount of interior mesh 16 to an amount sufficient to enable one-way moisture transfer. As illustrated in FIG. 3, apron 22 is connected by a horizontal seam 24 to the remainder of the interior panel 21, which may comprise the interior mesh 16. In some implementations, the interior mesh 16 extends to the rear scam 38 of right gusset section 36a and left gusset section 36b. The right section of right gusset section 36a can be a strip of fabric on the inner right leg bordered by the following: the rear gusset scam 38, the front right gusset seam 40a, the right vertical seam 18a framing the interior panel 21, and the hem of the right underwear leg 42a. The left gusset section 36b can be a strip of fabric on the inner left leg bordered by the following: the rear gusset seam 38, the front left gusset seam 40b, the left vertical seam 18b framing the interior panel 21, and the hem of the left underwear leg 42b. The rear gusset seam 38 of the two gusset sections right gusset section 36a and left gusset section 36b can be placed so that the interior mesh 16 extends past the scrotum 26 (see FIG. 4) to the perineum 28 (see FIG. 4) of a wearer of the boxer briefs 10. In some implementations, the incontinence guard 12 and pouch 15 are designed such that the incontinence guard 12 fits securely in the pouch 15. When incontinence guard 12 is inserted into the pouch 15, the incontinence guard 12 is held securely in place by the right vertical seam 18a, left vertical seam 18b, the rear gusset seam 38, the exterior front panel 30 (see FIG. 2), and the interior mesh 16. In some implementations, the incontinence guard 12 can be sized long enough that it extends from a short distance below the top edge 32 of the exterior front panel 30 (see FIG. 2) to the rear gusset seam 38. In some implementations, the interior mesh 16 lies against the wearer's genitalia. In some implementations, the interior mesh 16 is in communication with the wearer's skin to increase the moisture that is drawn from the wearer's skin. When the wearer urinates, the urine can be efficiently conducted by the interior mesh 16 from the penis 17 (see FIG. 4) into the incontinence guard 12. In some implementations, the removable absorbent incontinence guard 12 can be disposable or reusable.

FIG. 4 illustrates some implementations of a pair of boxer briefs 10 that include a pouch 15. The pouch 15 can be configured to hold the removable absorbent incontinence guard 12. The pouch is formed by an interior panel 21 and an exterior front panel 30. In some implementations, the interior panel 21 can be divided into two subsections that are connected: an apron 22, which can be made from the main body fabric of the underwear and a second fabric section, such as the interior mesh 16. In some implementations, the apron 22 extends down from the waistband 23 for a small portion of the length of the interior panel 21. For example, in some implementations, apron 22 may extend down approximately 3 inches from waistband 23. It should be appreciated that the exact dimensions of apron 22 may vary depending on the size of the boxer briefs 10. In some implementations, apron 22 is connected by a horizontal seam 24 to interior mesh 16 to form the interior panel 21. In some implementations, the exterior front panel 30 and the interior mesh 16 extend to the rear gusset seam 38 of the left gusset section 36b. The left gusset section 36b can be a strip of fabric on the inner left leg bordered by the following: the rear gusset seam 38, the left front gusset seam 40b, the left vertical seam 18b framing the interior panel 21, and left leg hem 42b. The rear gusset seam 38 of the left gusset section 36b can be placed so that the interior mesh 16 extends past the scrotum 26 to the perineum 28. The top edge 32 of the exterior front panel 30, located a short distance below the waistband 23 can be left unconnected to the main body of the underwear garment to create the pouch opening 34 of the pouch 15. In some implementations, top edge 32 can be placed substantially in line with the edge of waistband 23 or in some other suitable position below or above waistband 23. This pouch opening 34 is configured to removably receive the incontinence guard 12. The incontinence guard 12 and pouch 15 are designed such that the incontinence guard 12 fits securely in the pouch. In some implementations, incontinence guard 12 is prevented from substantial side to side movement (e.g., along a horizontal axis that is perpendicular with waistband 23). When incontinence guard 12 is inserted into the pouch 15, the incontinence guard 12 can be held securely in place by the two vertical seams right vertical seam 18a and left vertical seam 18b (see FIGS. 2, 3, and 5), the rear gusset seam 38, the exterior front panel 30, and the interior mesh 16. In some implementations, the incontinence guard 12 can be sized long enough that it extends from a short distance below the top edge 32 of the exterior front panel 30 to the rear gusset seam 38. In some implementations, the interior mesh 16 is in at least partial communication with the wearer's genitalia to promote capture of urine. In some implementations, the interior mesh 16 is in communication with the wearer's skin to increase the moisture that is wicked or otherwise drawn from the wearer's skin. When the wearer releases urine, the urine can be efficiently conducted by the interior mesh 16 from the penis 17 into the incontinence guard 12. In some implementations, the exterior front panel of body fabric 30 can be formed from waterproof or water-resistant material to further reduce or prevent moisture from seeping from the incontinence guard 12 or interior mesh 16 to the exterior of the boxer briefs 10. In some implementations, the removable absorbent incontinence guard 12 can be disposable or reusable.

FIG. 5 illustrates some implementations of a pair of boxer briefs 10 that include a pouch 15. The pouch 15 can be configured to hold a removable absorbent incontinence guard 12. The pouch is formed by an interior panel 21 and exterior front panel 30 (see FIG. 2). The interior panel 21 is attached to two vertical seams right vertical seam 18a and left vertical scam 18b that frame the interior panel 21. In some implementations, the interior panel 21 is configured to be wide enough to cover the genitalia of the wearer, with some extra margin to account for movement of the boxer briefs 10 or garment when the wearer is sitting or standing. In some implementations, the interior panel 21 can be divided into two subsections that are connected: an apron 22 made from the main body fabric of the underwear and at least one second panel of fabric, such as the interior mesh 16. In some implementations, the apron 22 extends down from the waistband 23 for a small portion of the length of the interior panel 21. Apron 22 is connected by a horizontal seam 24 to the interior mesh 16 to form the interior panel 21. It should be appreciated that in some implementations, interior panel 21 may include more or fewer segments of fabric. In some implementations, interior panel 21 can be formed from one segment. In some implementations, the one segment can be mesh fabric or some other suitable material. In some implementations, the interior panel 21 can be formed from one or more layers. As will be described in greater detail below, in some implementations, different layers of material can work together to draw moisture away from a wearer's body and aid in the rapid transfer of the moisture to the incontinence pad 12. In some implementations, the interior mesh 16 extends to the rear gusset seam 38 of the right gusset section 36a and left gusset section 36b. The right gusset section 36a can be a strip of fabric on the inner right leg bordered by the following: the rear gusset seam 38, the right front gusset seam 40a, the right vertical seam 18a framing the interior panel 21, and the right leg hem 42a. The left gusset section 36b can be a strip of fabric on the inner left leg bordered by the following: the rear gusset seam 38, the left front gusset seam 40b, the left vertical seam 18b framing the interior panel 21, and the hem of the left underwear leg 42b. The rear gusset seam 38 of the two gusset sections right gusset section 36a and left gusset section 36b can be placed so that the interior mesh 16 extends past the scrotum 26 (see FIG. 4) to the perineum 28 (see FIG. 4). The incontinence guard 12 and pouch 15 can be designed such that the incontinence guard 12 fits securely in the pouch 15. When it is inserted into the pouch 15, the incontinence guard 12 can be held securely in place by the two vertical seams right vertical seam 18a and left vertical seam 18b, the rear gusset scam 38, the exterior front panel 30 (scc FIG. 2), and the interior mesh 16. In some implementations, the incontinence guard 12 can be sized long enough that it extends from a short distance below the top edge of the exterior front panel 30 (see FIG. 2) to the rear gusset scam 38. The interior mesh 16 lies at least partially against the wearer's genitalia. In some implementations, the interior mesh 16 is in communication with the wearer's skin to increase the moisture that is wicked or otherwise drawn from the wearer's skin. When the wearer produces urine while wearing the boxer briefs 10, the urine can be efficiently conducted by the interior mesh 16 from the penis 17 (scc FIG. 4) into the incontinence guard 12 until the incontinence guard 12 can be changed. In some implementations, the removable absorbent incontinence guard 12 can be disposable or reusable.

In various implementations, the incontinence guard 12 can be sized differently for different applications. In some implementations, the edges of the incontinence guard 12 may not substantially be in communication with one or more of the two vertical seams right vertical scam 18a and left vertical seam 18b. In some implementations, the incontinence guard 12 may have one or more of the following attributes: In order to fit securely in the pouch 15 of different sizes (for different sizes of boxer briefs 10, the incontinence guard 12 may be provided in a range of sizes. The sizes of the incontinence guard 12 may range from XS (extra-small) to XXL (double extra-large) in order to correspond with offered sizes of garments having a pouch 15 such as described herein to hold the incontinence guard 12. In some implementations, the top sheet of the incontinence guard 12 may be configured with a range of colors, designs, or a combination of the forgoing so as to match the color of the garment with the pouch 15 that holds the incontinence guard 12 and therefore be less conspicuous. In some implementations, exterior front panel 30 can be constructed from a substantially opaque fabric to prevent the incontinence guard 12 from being visible. In some implementations, the incontinence guard 12 has a waterproof backsheet made from breathable nonwoven material. In some implementations, the incontinence guard 12 comprises a backsheet made from other suitable materials. In some implementations, the backsheet of the incontinence guard 12 does not have an adhesive section or release paper. In some implementations, the incontinence guard 12 lacks other sorts of fastener to secure the incontinence guard 12 to the pair of boxer briefs 10. That is, the incontinence guard 12 is provided independent of a fastener to secure the incontinence guard 12 to the pair of boxer briefs 10. By not using an adhesive section on the incontinence guard 12, a wearer can more easily place and remove the incontinence guard 12 from the pouch 15. In some implementations, the incontinence guard 12 is disposable. In some implementations, the incontinence guard 12 comprises a shape that is the same or similar to the pouch 15 to enable the incontinence guard 12 to better fit and stay within pouch 15.

In some implementations, the body fabric of the boxer briefs 10 may be a knit fabric with 4-way stretch. Other suitable types of fabric can be used as the body fabric for boxer briefs 10. As noted herein, in some implementations, boxer briefs 10 may comprise two or more types of body fabric. It should be appreciated that modal, polyester/elastane, nylon, wool, silk, bamboo rayon, cotton, viscose, and other similar fabrics may be used alone or in combination for the underwear.

In some implementations, the interior mesh 16 may be a small hole mesh (a.k.a. “gauze mesh”) made of polyester, nylon, elastane, or some combination thereof. In some implementations, the interior mesh 16 is designed to transfer at least some or substantially all of the urine into the incontinence guard 12, with very little moisture staying in the interior mesh 16. In some implementations, the interior mesh 16 enables one-way moisture transfer from the human body to the incontinence guard 12 and substantially prevents moisture from remaining between the human body and the mesh 16. In some implementations, the hole size of interior mesh 16 (such as mesh openings 606) enables faster transport of moisture from the wearer's body to the incontinence guard 12. In some implementations, the one-way moisture transport enables the interior mesh 16 to feel dry to the wearer, even if the wearer experiences multiple urine leaks in a short period of time. In some implementations, the interior mesh 16 may be treated with a chemical solution designed to minimize odors. For example, interior mesh 16 can be treated with antimicrobial solution which prohibits microbe growth. In some implementations, interior mesh 16 can be treated an odor absorption solution, which helps interior mesh 16 absorb and trap unwanted odors, such as odors that originate from sweat. In addition, in some implementations, the interior mesh 16 may be treated with a chemical solution designed to mitigate urine odors by reducing or neutralizing ammonia, the primary odor-causing chemical in urine. In some implementations, the interior mesh 16 may be further treated with a chemical solution that further enhances the hydrophilic qualities of the fabric of interior mesh 16 to more effectively pull moisture away from the body to the incontinence guard 12. In some implementations, a wearer can apply or reapply additional chemical solutions to maintain or return the hydrophilic qualities of the fabric of interior mesh 16.

In some implementations, the fabric used for interior mesh 16 can be configured to provide substantial one-way moisture transport from the wearer's body to the incontinence guard 12 with or without chemical treatment. In some implementations, the fabric used for interior mesh 16 can be configured to provide wicking moisture from a wearer's body and efficiently transfer wicked moisture to the incontinence guard 12 with or without chemical treatment. In some implementations, the interior mesh 16 can be configured with one side that is hydrophilic and one side that is hydrophobic. In some such implementations, interior mesh 16 may comprise more than one layer of fabric, wherein one of the fabric layers is hydrophilic and one side that is hydrophobic. In some implementations, the hydrophilic layer can be placed in communication with the wearer's skin and can wick away any fluids to transport the fluids to hydrophobic layer, which can then transport the fluids to the incontinence guard 12. In some implementations, the fabric for interior mesh 16 can be constructed with a tighter knit on one side and a looser knit on the other side. The tighter knit can be in communication with the wearer's skin and the looser knit can be communication with the incontinence guard 12. The tighter knit of interior mesh 16 more readily draws moisture away from the wearer's body and the looser knit allows the moisture to spread on the surface of interior mesh 16 when in communication with the incontinence guard 12 to enable faster transport of moisture to the incontinence guard 12. In some implementations, interior mesh 16 may comprise different yarn sizes on either side. A smaller yarn size or diameter may encourage faster wicking of moisture from the wearer's body while the larger and looser yarn enables the moisture to spread out for faster transport to the incontinence guard 12.

As noted above, although interior mesh 16 is described as a mesh fabric, in some implementations, the interior panel 21 does not use a mesh fabric where the interior mesh 16 is located. In some implementations, an alternative fabric with suitable rapid and substantially one-way moisture transport that is can be used as a substitute for the mesh fabric.

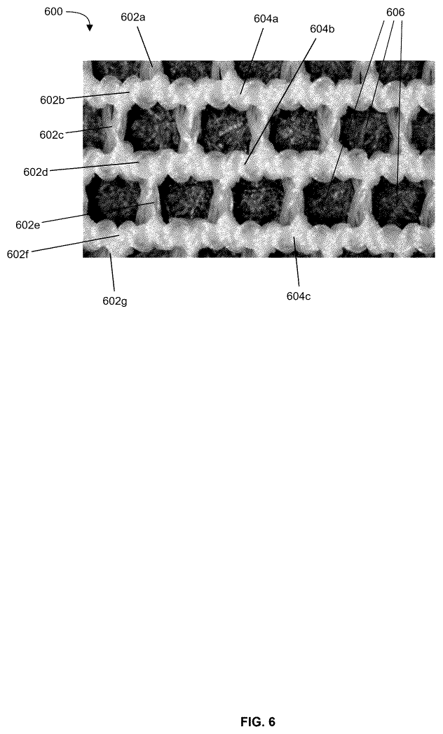

Referring to FIG. 6, a mesh knit structure 600 may be constructed using multiple interconnected yarn strands. In some implementations, the mesh knit structure 600 can be constructed using a warp knit. In other implementations, a different knit can be used. In some cases, the mesh knit structure 600 may be used in the construction of the boxer briefs 10, such as for the interior mesh 16. The mesh knit structure 600 may comprise multiple yarn strands that run vertically through the fabric structure and are interconnected with a warp knit. In some implementations, the warp knit may include wrapping yarn strands with a specific wrapping pattern around horizontal opposed elastane fibers. Yarn strands may be formed from multiple continuous yarn fibers (such as bundles of yarn fibers) that work together.

In some implementations, the mesh knit structure 600 comprises multiple interconnected yarn strands. Using one yarn strand as an example, yarn strand portion 602a, yarn strand portion 602b, yarn strand portion 602c, yarn strand portion 602d, yarn strand portion 602c, yarn strand portion 602f, and yarn strand portion 602g form a portion of a single yarn strand that is shown running from the top of the mesh knit structure 600 to the bottom of the mesh knit structure 600. It should be appreciated that in some implementations, the yarn strand formed from yarn strand portions 602a-602g may comprise multiple continuous yarn fibers. In some implementations, such yarn strand portions are wrapped over one or more strands of other yarn or fibers. As illustrated, yarn strand portions 602a-602f are wrapped around one or more elastane fibers or elastane fiber bundles such as elastane fiber 604a, elastane fiber 604b, and elastane fiber 604c, which can be horizontally opposed to the yarn strands. For example, yarn strand portion 602a can run vertically from the top of the image of mesh knit structure 600 and can be wrapped around the top elastane fiber 604a to the left of yarn strand portion 602a as shown with yarn strand portion 602b. The yarn strand can be run vertically down as shown with yarn strand portion 602c to the next elastane fiber 604b. The yarn strand can be wrapped around the elastane fiber 604b to the right of yarn strand portion 602c, as illustrated with yarn strand portion 602d. The yarn strand portion 602e can run vertically down to the next elastane fiber 604c and can be wrapped around the elastane fiber 604c to the left of yarn strand portion 602e as shown with yarn strand portion 602f. Yarn strand portion 602g shows the continuation of the pattern with the yarn strand running out of the illustration.

In some cases, multiple such yarn strands, such as the yarn strand formed from yarn strand portions 602a, 602b, 602c, 602d, 602c, 602f, and 602g may be positioned parallel to one or more other yarn strands across the width of the mesh knit structure 600. In some implementations, the one or more yarn strands may follow the same or similar alternating wrapping pattern described above around horizontal elastane fibers. The yarn strands may be knit together in both horizontal and vertical directions, intersecting at regular intervals to form the structural framework of the mesh knit structure 600. This knitting pattern may create a network of mesh openings 606 that can be arranged in a grid-like configuration throughout the mesh knit structure 600. It should be appreciated that the mesh knit structure 600 is merely an illustration of a small portion of a mesh knit fabric. A warp knit pattern, such as illustrated in FIG. 6, can be repeated across any suitable width and length to form the mesh fabric.

The mesh openings 606 may be formed by the spaces created between the intersecting yarn strands and horizontal elastane fibers. In some cases, the size and shape of the mesh openings 606 may be determined by the spacing between the yarn strands and the tension applied during the knitting process. The mesh openings 606 may allow for more efficient one-way fluid transfer through the mesh knit structure 600. A mesh knit structure 600 may encompass the overall three-dimensional configuration of the mesh knit structure 600, including the yarn strand portions and the mesh openings 606. The mesh knit structure 600 may provide the functional characteristics used for moisture wicking and fluid transfer when used as the interior mesh 16 of the boxer briefs 10.

The alternating wrapping patterns of the yarn strand portions around the horizontal elastane fibers may create a stable and flexible fabric structure that can stretch and recover while maintaining the integrity of the mesh openings 606. In some cases, the yarn strand portions may be made from materials such as polyester, nylon, or other synthetic fibers that provide durability and moisture-wicking properties. The horizontal elastane fibers may provide stretch and recovery characteristics to the mesh knit structure 600, allowing the fabric to conform to body movements while maintaining the proper positioning of the mesh openings 606 for optimal fluid transfer performance.

The present disclosure is not to be limited in terms of the particular implementations described in this disclosure, which are intended as illustrations of various aspects. Moreover, the various disclosed implementations can be interchangeably used with each other, unless otherwise noted. Many modifications and variations can be made without departing from its spirit and scope, as will be apparent to those skilled in the art. Functionally equivalent methods and apparatuses within the scope of the disclosure, in addition to those enumerated herein will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used herein is for the purpose of describing particular implementations only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to implementations containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” In addition, where features or aspects of the disclosure are described in terms of Markush groups, those skilled in the art will recognize that the disclosure is also thereby described in terms of any individual member or subgroup of members of the Markush group. A number of implementations have been described. Various modifications may be made without departing from the spirit and scope of the disclosure. Accordingly, other implementations are within the scope of the following claims.

A number of implementations have been described. Nevertheless, it will be understood that various modifications may be made without departing from the spirit and scope of the disclosure. Accordingly, other implementations are within the scope of the following claims.

I claim:

1. A garment comprising:
a gusset;
a pouch, wherein the pouch comprises:
a front panel;
a rear panel, wherein the rear panel comprises a fabric that is different from other parts of the garment;
two vertically opposed seams formed between the front panel and the rear panel;
a horizontally opposed lower seam formed between the front panel, the rear panel, and the gusset;
an opening formed between the front panel and the rear panel; and
a removable incontinence guard, wherein the removable incontinence guard is positioned within the pouch,
wherein the opening of the pouch is positioned on an outside of the garment.

2. The garment of claim 1, wherein the fabric that is different from other parts of the garment is a mesh fabric.

3. The garment of claim 2, wherein the mesh fabric is treated to be hydrophobic and efficiently transports body fluids from one side of the mesh fabric to the other side of the mesh fabric.

4. The garment of claim 3, wherein one side of the mesh fabric is in at least partial communication with a human body and the mesh fabric transports body fluids from the human body.

5. The garment of claim 1, wherein the garment comprises underwear.

6. The garment of claim 5, wherein the garment comprises boxer briefs.

7. The garment of claim 5, wherein the garment comprises briefs.

8. The garment of claim 1, wherein the garment comprises workout pants or shorts.

9. The garment of claim 1, wherein the pouch further includes a securing means positioned near the opening of the pouch.

10. The garment of claim 9, wherein the pouch further includes an elastic band to narrow the opening of the pouch to removably retain the incontinence guard within the pouch.

11. The garment of claim 1, wherein the front panel is substantially a same color as the removable incontinence guard.

12. The garment of claim 1, wherein the front panel substantially prevents moisture from passing through the front panel.

13. The garment of claim 1, wherein the fabric that is different from other parts of the garment is a provides a one-way transport of moisture away from a human body to the removable incontinence guard.

14. The garment of claim 1, wherein the front panel comprises a reinforced lip at the opening.

15. The garment of claim 1, wherein the front panel comprises two panels of fabric.

16. The garment of claim 1, wherein the pouch is formed on the outside of the garment.

17. The garment of claim 1, wherein the opening is located at the top of the garment.

18. The garment of claim 1, wherein the removable incontinence guard is provided independent of a fastener to secure the removable incontinence guard inside the pouch.

19. The garment of claim 1, wherein the pouch is tapered such that the top of the pouch is wider than the bottom of the pouch.

20. The garment of claim 19, wherein the removable incontinence guard is tapered to substantially match with the taper of the pouch.
